(19) United States
(12) Patent Application Publication
(10) Pub. No.: US 2007/0207503 A1
(43) Pub. Date: Sep. 6, 2007

(54) DETECTION OF ALCOHOL-ESTERIFIED FATTY ACIDS

(76) Inventors: Hyesook Kim, Bloomfield Hills, MI (US); Elizabeth S. Roberts-Kirchoff, New Baltimore, MI (US)

Correspondence Address: KOHN & ASSOCIATES, PLLC, 30500 NORTHWESTERN HWY, STE 410, FARMINGTON HILLS, MI, 48334, US

(21) Appl. No.: 11/681,243

(22) Filed: Mar. 2, 2007

Related U.S. Application Data
(60) Provisional application No. 60/779,086, filed on Mar. 3, 2006.

(51) Int. Cl. G01N 33/53; C12P 21/04; C12P 7/64

(52) U.S. Cl. 435/7.1; 435/70.21; 435/134

(57) ABSTRACT

A method of producing an antibody recognizing a target fatty acid alcohol ester by immunizing an animal with a carrier protein conjugated with a derivative of the target fatty acid alcohol ester. A method of estimating alcohol consumption of an individual by quantitating levels of molecules which bind to antibodies produced with a derivative of a target fatty acid alcohol ester conjugated to a carrier protein in a biological sample obtained from the individual. A kit for estimating alcohol consumption of an individual including means for quantitating levels of molecules which bind to antibodies produced with a derivative of a target fatty acid alcohol ester conjugated to a carrier protein in a biological sample obtained from the individual.

### CROSS-REFERENCE TO RELATED APPLICATIONS

This application claims benefit under 35 USC §119 (e) of United States Provisional Patent Application Ser. No. 60/779,086, filed Mar. 3, 2006.

### GOVERNMENT SUPPORT

Research in this application was supported in part by a grant from the National Institute on Alcohol Abuse and Alcoholism (R43 AA014535).

### BACKGROUND OF THE INVENTION 1. Field of the Invention

The present invention relates to the detection of alcohol use. In particular, the present invention relates to antibodies of fatty acid alcohol esters

2. Description of Related Art

Alcoholism or alcohol dependence is an illness of compulsive heavy consumption of alcoholic beverage which develops withdrawal symptoms. One out of fifty in the US population has an alcohol dependence problem which induces alcohol-related diseases including liver and heart disease and nervous system disorders.

Fetal alcohol syndrome (FAS) is a pattern of growth retardation, characteristic facial anomalies and mental retardation in children born to alcoholic women. Also at risk are offspring exposed prenatally to moderate levels of alcohol or occasional maternal abuse, especially binge drinking. Exposures to low or moderate levels of alcohol show dose-dependent and distinguishing patterns of cognitive dysfunction (1, 2). Perinatal alcohol consumption is the most common preventable cause of mental retardation in the developed world (3, 4).

It is essential to begin remedial treatment of FAS children as early as possible in order to affect an optimal outcome (3). However, delivery of the needed services and medical care is complicated by a need to target children with the highest risk. Determination of alcohol use by self-report of a mother is not reliable. Moreover, although FAS is a result of maternal alcohol intake, maternal drinking is not always detrimental to the offspring, even when the mother is a serious alcoholic (5). Because resources are limited and remedial treatments are costly, it would be extremely useful to identify before or at birth infants exposed prenatally to alcohol that will be negatively impacted so medical and sociological efforts could be directed specifically to those most in need.

One of biomarkers which correlates the prenatal exposure of an infant to alcohol is fatty acid ethyl ester level in meconium (6, 7). So far levels of fatty acid ethyl esters in meconium have been measured by gas chromatography/mass spectroscopy (GC/MS) or gas chromatography/flame ionization detection (GC/FID) which is tedious and requires an extensively trained technician and expensive instruments.

Thus, facile methods are needed to detect fatty acid ethyl esters for the diagnosis of FAS and other alcohol related conditions.

### SUMMARY OF THE INVENTION

The present invention provides for a method of producing an antibody recognizing a target fatty acid alcohol ester by immunizing an animal with a carrier protein conjugated with a derivative of the target fatty acid alcohol ester.

The present invention further provides for a method of estimating alcohol consumption of an individual by quantitating levels of molecules which bind to antibodies produced with a derivative of a target fatty acid alcohol ester conjugated to a carrier protein in a biological sample obtained from the individual.

The present invention also includes a kit for estimating alcohol consumption of an individual including means for quantitating levels of molecules which bind to antibodies produced with a derivative of a target fatty acid alcohol ester conjugated to a carrier protein in a biological sample obtained from the individual.

### DESCRIPTION OF THE DRAWINGS

Other advantages of the present invention will be readily appreciated as the same becomes better understood by reference to the following detailed description when considered in connection with the accompanying drawings wherein:

FIG. 1 is a graph showing limitation of immunogenic site length of a fatty acid for form-specific antibody production. Form-specific antibody productions of unique dihydroxyl sites of 14,15- and 11,12-DHETs, which are located at least 10 carbons away from the carrier protein, were successful whereas form-specific antibody productions of unique dihydroxyl sites of 8,9- and 5,6-DHETs, which are located 7 and 5 carbons, respectively, away from the carrier protein, failed.

FIG. 2 is a graph showing enzymatic production of oxygenated metabolites of linoleic and arachidonic acids.

FIGS. 3A and 3B are graphs showing structures of ethyl esters (underlined) of linoleic acid and a biological metabolite 13-HODE (Panel A) or ethyl ester (underlined) of arachidonic acid and a biological metabolite 20-hydroxy arachidonic acid (20-HETE) (Panel B). Ethyl esters of 13-HODE were conjugated to keyhole limpet hemocyanin (KLH) (immunization), bovine serum albumin (BSA) (screening) and horseradish peroxidase (HRP) (ELISA) via the OH group at 13C. The ethyl ester of 13-HODE differs from the ethyl ester of linoleic acid only in 3 carbons located close to the OH group of the 13C which is not easily accessible to solution when the ethyl ester of 13-HODE is conjugated to KLH. Unique antigenic sites of the fatty acids which are at least 10 carbons (5 of the 10 carbons are from succinate derivatization via the OH group) away from the carrier proteins are in bold type.

FIGS. 4A and 4B are graphs showing a GC/MS chromatogram of 13-HODE ethyl ester succinate derivative (Panel A) and a mass spectrum of 13-HODE ethyl ester succinate derivative (Panel B). In Panel A, 13-HODE ethyl ester succinate derivative is marked with an arrow.

FIGS. 5A, 5B, and 5C are graphs showing ELISA, SDS-PAGE and Western blot analysis, respectively, of BSA (A) or BSA-ethyl ester of 13-HODE (B). Cross-reactivity of antibodies produced by immunization of a goat with 13-HODE ethyl ester conjugated KLH was determined by an ELISA and Western blot analysis. In ELISA (5A), BSA and 13-HODE ethyl ester conjugated BSA were coated on plates. After incubation of the plate with the primary antibodies, bound antibodies were visualized by an anti-goat-HRP secondary/TMB HRP substrate system. In Western blot analysis, BSA and 13-HODE ethyl ester conjugated BSA were separated by SDS-PAGE (5B), transferred to nitrocellulose membrane and Western blot analysis (5C) was carried out by visualizing bound primary antibody with an anti-goat-HRP secondary/ECL system.

FIG. 6 is a graph showing a competitive ELISA carried out with various concentrations of BSA and 13-HODE ethyl ester conjugated BSA to show cross-reactivity of the antibody to the 13-HODE ethyl ester.

### DETAILED DESCRIPTION OF THE INVENTION

The present invention provides generally for a method of producing an antibody that recognizes a target fatty acid alcohol ester. Throughout this application, the word “recognizing” is synonymous with “binding”. In other words, the antibody binds with the target fatty acid alcohol ester. The antibodies are produced by immunizing an animal with a carrier protein conjugated with a derivative of the target fatty acid alcohol ester. It was previously unknown where form-specific alcohol esterified fatty acid antibody production occurred utilizing derivatized fatty acid ethyl esters.

As the present invention details herein, limitations of the immunogenic site length of a fatty acid for production of form-specific antibody production have been estimated by producing antibodies for 4 DHETs (20 carbon-fatty acids) which are products obtained by biotransformation of the arachidonic acid by catalysis of cytochromes P450 and epoxide hydrolase.

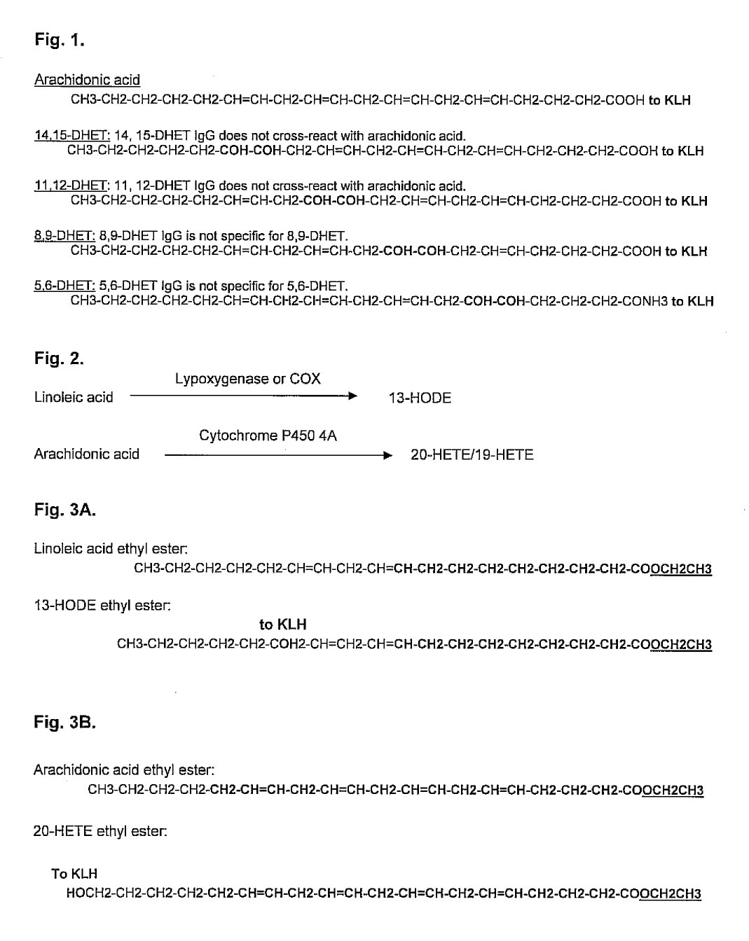

The 4 fatty acids have an identical molecular structure except for a unique dihydroxyl group located at C14-C15 (14,15-DHET), C11-C12 (11,12-DHET), C8-C9 (8,9-DHET) and C5-C6 (5,6-DHET). The hydroxyl derivative of the target fatty acid is obtained by enzymatic or chemical oxidation of the target fatty acid followed by selection of an appropriate hydroxyl derivative of the target fatty acid. Form-specific antibody productions for 14,15- and 11,12-DHETs were successful whereas form-specific antibody productions for 8,9- and 5,6-DHETs failed (FIG. 1). It is a surprise that antibody production of 8,9-DHET which has the dihydroxyl site close to that of 11,12-DHET failed. The result showed that the unique antigenic site of a fatty acid has to be located at least 10 carbons away from the carrier protein. Therefore, the present invention provides derivatives of target fatty acid alcohol esters including a unique antigenic site at least 10 carbons away from the carrier protein.

Usually fatty acid antibody production is carried out after conjugation of the fatty acids to a carrier protein such as KLH via the COOH at C1 of the fatty acids. Thus, blocking the COOH site by ethyl esterification leaves the fatty acids free of any available COOH group.

Antibodies were produced with a metabolite of the fatty acid which has an OH group at a suitable position away from the target moiety (i.e., the ethyl group) at C1. Transformation of the OH group to the COOH group prior to conjugation of the fatty acid to a carrier protein via the COOH group abolishes differences between the target fatty acid and the metabolite as far the antibody recognition site of the target molecule.

Utilizing this finding that any differences at and close to the conjugation site between the target and metabolite molecules do not alter the specificity of an antibody, antibodies for the linoleic acid ethyl ester were produced using the structurally similar 13-HODE ethyl ester. While specific esters were made herein, antibodies for any other fatty acid alcohol ester meeting the requirement of a unique antigenic site at least 10 carbons away from the carrier protein can be produced.

The 13-HODE ethyl ester is a metabolite of linoleic acid by catalysis of lipoxygenase or prostaglandin H₂ synthase (PGHS) or also called as cyclooxygenase (COX) (FIG. 2) and is structurally same as linoleic acid except for an OH group at C13 and a double bond at C11-C12 (linoleic acid has no OH group at C13 and a double bond at C12-C13) (FIG. 3, Panel A).

This method can be used for production of antibodies for other fatty acid ethyl esters including arachidonic acid ethyl ester. 20-Hydroxy arachidonic acid (20-HETE), a metabolite of arachidonic acid by catalysis of cytochrome P450 4A (FIG. 2), which has an OH group at C20, can be used for antibody production of arachidonic acid (FIG. 3B). The 20-HETE can be conjugated to KLH via the COOH group at 20C which is obtained after transformation of the OH group at 20C.

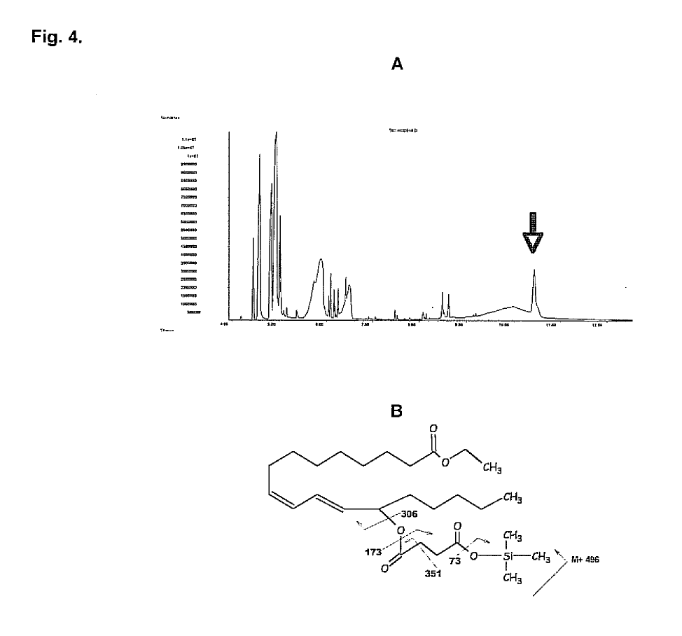

13-HODE, a metabolite of linoleic acid, was converted to an ethyl ester with lipase (Candida antarctica) acrylic resin and ethyl alcohol in acetone (8) with modification. The ethyl ester was converted to a succinate derivative via its C13 hydroxyl group in the presence of succinic anhydride with 4-dimethylaminopyridine (4-DMAP) as a catalyst in chloroform under an argon atmosphere over 2 days (9). GC/MS analysis was performed after derivatization with MSTFA (FIG. 4). The samples were analyzed on a HP 6890 Series GC system with a quadrupole EI/MS detector at 70 eV and a HP-5MS (capillary 30.0 m×250 μm×0.25 μm nominal 5% phenyl methyl siloxane) column. The injector temperature was 250° C. for 2 minutes, with 30° C./minute increased to 325° C. for 5 minutes. The software program was Enhanced ChemStation. The succinate derivative has the O—CO—CH2-CH2-COOH group attached at C13.

The 13-HODE ethyl ester succinate derivative in a mixture of other products, as shown in FIG. 4, Panel A, was cross-linked to KLH, BSA and HRP via the O—CO—CH2-CH2-COOH group at C13 (10). By this conjugation method, the distance from C13 to a carrier protein is a 5 carbon length. The KLH derivative was used to immunize a goat by Cocalico Biologicals. Titers of bleeds were tested by ELISA with ethyl fatty acid-BSA conjugates.

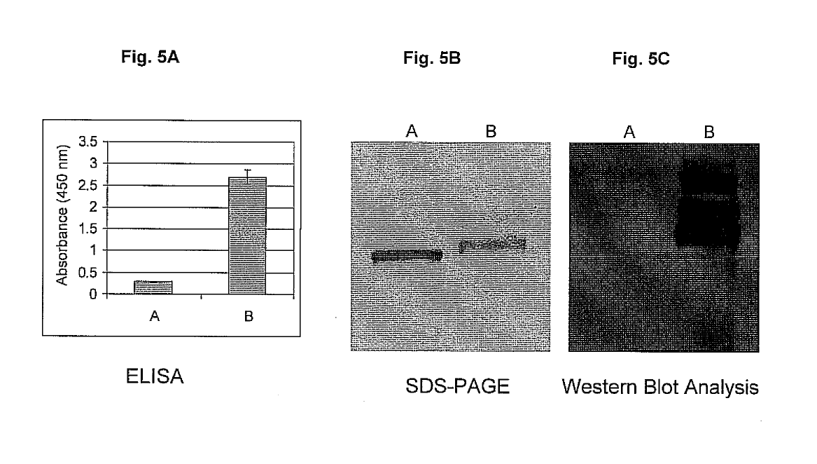

When BSA or BSA conjugated with ethanol-esterified 13-HODE (10 μg/well) were coated on a plate and hybridized with ethanol-esterified 13-HODE IgG followed by hybridization of the plate with anti-goat IgG/HRP and visualization of the plate with a HRP substrate, the well coated with BSA conjugated with ethanol-esterified 13-HODE showed ˜9-fold high absorbance at 450 nm compared with BSA-coated well (FIG. 5A).

Net absorbance at 450 nm, obtained by subtracting absorbance of BSA-coated well from absorbance of the well coated with BSA conjugated with ethanol-esterified 13-HODE, correlated with amounts of the succinate product (see the arrow in FIG. 4). The 0.9×105 and 29×105 units of the succinate products correlated with optical densities (ODs) of 0.51 and 0.99, respectively, by ELISA (Table 1). Approximately 2-fold difference in the ODs represents much higher difference of bound ethanol-esterified 13-HODE IgG because of the curved graph of absorbances vs. succinate product concentrations (usually an exponential curve produces a straight line). This result demonstrates that antibodies recognize primarily the ethanol-esterified 13-HODE.

Western blot analysis with 20 μg/lane of BSA or BSA conjugated with ethanol-esterified 13-HODE revealed that, whereas the IgG did not bind to BSA, the IgG strongly bound to BSA conjugated with ethanol-esterified 13-HODE (FIG. 5C).

These results demonstrate that antibodies for ethanol-esterified 13-HODE, which also recognized ethanol-esterified linoleic acid, were successfully produced.

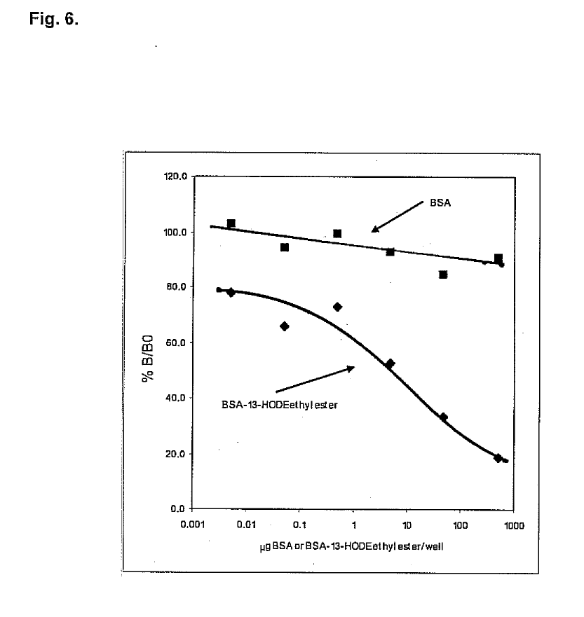

A competitive ELISA using a plate coated with the IgG was carried out with various concentrations of BSA conjugated with ethanol-esterified 13-HODE and HRP conjugated with ethanol-esterilied 13-HODE. A negative control for this ELISA was BSA. Whereas BSA did not compete with ethanol-esterified 13-HODE-HRP conjugate for binding to the IgG, BSA conjugated with ethanol-esterified 13-HODE competed with the HRP conjugate in a dose-dependent manner (FIG. 6). Ethanol-esterified linoleic acid and ethanol also competed with the HRP conjugated with ethanol-esterified 13-HODE in a dose-dependent manner. The results demonstrate that competitive ELISA for quantitation of ethanol-esterified 13-HODE and ethanol-esterified linoleic acid was successfully produced.

In general the quantification of the sample is done utilizing an immunoassay as described in the Examples herein. Most of the techniques used in performing immunoassays are widely practiced in the art, and most practitioners are familiar with the standard resource materials which describe specific conditions and procedures. However, for convenience, the following paragraph may serve as a guideline.

In general, ELISAs are the preferred immunoassays employed to assess the amount of ethanol-esterified fatty acids in a specimen. ELISA assays are well known to those skilled in the art. Polyclonal, monoclonal and recombinant antibodies can be used in the assays. Where appropriate other immunoassays, such as radioimmunoassays (RIAs) or fluoroimmunoassays (FIAs) can be used as are known to those in the art. Available immunoassays are extensively described in the patent and scientific literature. See, for example, U.S. Pat. Nos. 3,791,932; 3,839,153, 3,850,752; 3,850,578; 3,853,987; 3,867,517; 3,879,262; 3,901,654; 3,935,074; 3,984,533; 3,996,3451 4,034,074; 4,098,876; 4,879,219; 5,011,771 and 5,281,521 and may be adapted to be used the method of the present invention.

Ethanol-esterified fatty acids are measured utilizing the immunoassay as set forth for example in the Examples herein with an antibody which recognized esterified ethanol moiety of the ethanol esterified fatty acids. Alternatively, antibodies can be utilized to capture ethanol-esterified fatty acids followed by cleavage of the ester bond to release ethanol molecules which can be measured by HPLC or mass spectroscopy. Such antibodies can be produced as described herein and tested as set forth in Example 2.

Most of the techniques used to produce antibodies are widely practiced in the art, and most practitioners are familiar with the standard resource materials which describe specific conditions and procedures. However, for convenience, the following paragraphs may serve as a guideline.

Antibody production: Antibodies (immunoglobulins) may be either monoclonal or polyclonal and are raised against the immunogen. Such immunogens can be used to produce antibodies by standard antibody production technology well known to those skilled in the art as described generally in Harlow and Lane, Antibodies: A laboratory Manual, Cold Spring Harbor Laboratory, Cold Spring Harbor, N.Y., 1988 (11) and Borrebaeck, Antibody Engineering—A practical Guide, W. H. Freeman and Co., 1992 (12). Antibody fragments may also be prepared from the antibodies and include Fab, F(ab′)², and Fv by methods known to those skilled in the art.

For producing polyclonal antibodies a host, such as a rabbit or goat, is immunized with the immunogen, generally together with an adjuvant and, if necessary, coupled to a carrier antibodies to the immunogen are collected from the sera. Further, the polyclonal antibody can be absorbed such that it is specific for ethyl ester moiety of the ethyl esterified fatty acids. That is, the sera can be absorbed against related immunogens, e.g. the free fatty acids without an ethyl moiety or fatty acids conjugated with other OH group-containing molecules such as phenol-esterified fatty acids, so that no antibodies cross-reactive to the fatty acid moiety of the ethyl esterified fatty acids remain in the sera thereby rendering it monospecific antibodies for ethyl esters.

For producing monoclonal antibodies the technique involves hyperimmunization of an appropriate donor with the immunogen or immunogen fragment, generally a mouse, and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

For producing recombinant antibody (13-15), messenger RNAs from antibody producing B-lymphocytes of animals, or hybridoma are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody.

The antibody or antibody fragment can be bound to a solid support substrate or conjugated with a detectable moiety or be both bound and conjugated as is well known in the art to be used in the immunoassay (16). The binding of antibodies to a solid support substrate is also well known in the art (11,12). The detectable moieties contemplated with the present invention can include ferritin, alkaline phosphatase, β-galactosidase, peroxidase, urease, fluorescein, rhodamine, tritium, ¹⁴C and iodination as needed for the immunoassay.

The present invention further provides for a method of estimating alcohol consumption of an individual by quantitating levels of molecules which bind to antibodies produced with a derivative of a target fatty acid alcohol ester conjugated to a carrier protein in a biological sample obtained from the individual. An immunoassay can be performed as described above to quantitate the molecule levels. Preferably, the derivative of the target fatty acid alcohol ester includes a unique antigenic site at least 10 carbons away from the carrier protein. For example, the target fatty acid alcohol ester can be 13-HODE ethyl ester or 20-HETE ethyl ester as described above and in the examples below.

This method can be used to determine prenatal exposure of an infant to alcohol and the presence of FAS. In this case, the biological sample is mecodium obtained from the infant. Results from the immunoassay can be obtained in a much faster and more reliable way than questioning the mother of the infant.

Shortly after birth, a sample is-taken from the mecodium of an infant. An immunoassay such as ELISA is performed on the sample, and the results are analyzed to determine the amount of molecules bound to antibodies. The analysis is then used to determine if the infant was exposed to alcohol prenatally and aids in diagnosis of FAS.

This method can also be used to determine recent alcohol use by the individual. This is advantageous for recovering addicts or for anyone that should not ingest alcohol for a variety of reasons. While blood alcohol may not still be detectable after a certain amount of time, using this method to quantitate the levels of molecules can determine previous alcohol use.

Therefore, a biological sample can be obtained from an individual suspected of recent alcohol use. An immunoassay such as ELISA is performed on the sample, and the results are analyzed to determine the amount of molecules bound to antibodies. The analysis is then used to determine if the individual has recently ingested alcohol.

This method is also useful to determine if alcoholics are receiving successful treatment of their disease. For example, biological samples can be obtained and levels of molecules can be quantitated both before and after treatment of an individual with an alcohol-dependency lowering drug to estimate the effect of the drug. An immunoassay such as ELISA can be performed on the samples and analyzed for the amount of molecules bound to antibodies. This analysis is then used to determine if the alcohol-dependency lowering drug is successfully treating the individual's alcoholism.

The present invention also includes a kit for estimating alcohol consumption of an individual including an immunoassay for quantitating levels of molecules which bind to antibodies produced with a derivative of a target fatty acid alcohol ester conjugated to a carrier protein in a biological sample obtained from the individual. The kit generally includes a sample taking device such as a swab, syringe, or any other suitable device. The immunoassay can be those discussed above such as ELISA or any other suitable immunoassay. The kit can be prepackaged and available at hospitals, medical care facilities, emergency response units, police, or for individual use.

The above discussion provides a factual basis for the method of the present invention to measure ethyl esterified fatty acid as a profile of ethanol consumption of an individual. The elevated ethyl esterified fatty acids levels in a biological sample are a useful tool to develop a drug that lowers alcohol-dependency and monitor efficiency of the drug treatment. The methods used with and the utility of the present invention can be shown by the following non-limiting examples and accompanying figures.

### EXAMPLES

Materials and Methods

Materials

DHETs (higher than 98% pure by HPLC and GC/MS) were provided by Dr. Jorge Capdevila's laboratory. Horseradish peroxidase-conjugated donkey anti-goat immunoglobulin G (IgG) were purchased from Jakson ImmunoResearch Laboratories, Inc. (West Grove, Pa.). 15(S)HETE, 5(s)15(S)DiHETE, arachidonic acid, Thromboxane B₂, PGE2, PGF2_{α}, 6-keto-PGF_{1α} were obtained from Biomol Research Lab (Plymouth Meeting, Pa.). 13-HODE (higher than 98% pure by HPLC and GC/MS) was provided by laboratory of Dr. Art Bull at Oakland University. The ELISA kit was produced at Detroit R&D. Other reagents were obtained from Sigma Chemical Co.

Statistics

Statistical analysis was carried out using Statview 512 software (Brain Power, Inc., Calabasas, Calif.) and significance between groups was analyzed using one factor anova (Scheffe F-test).

### Example 1

### Development of the Immunoassay of 14,15-, 11,12-, 8,9- and 5,6-DHET

Antibody Production

Synthetic 14,15-, 11,12- or 8,9-DHETs were coupled to KLH using dicyclohexylcarbodiimide (DCC) as previously described (10). The 5,6-DHET with COOH at C1 blocked by NH3 to prevent lactone formation were coupled to KLH using 1-ethyl-3-(3-dimethylaminopropyl)carbodiimide hydrochloride (EDC) according to the manufacturer's instruction (Pierce Biotechnology, Rockford, Ill).

The conjugate was used to immunize a goat and antibody titers were determined by ELISA using the DHET-conjugated bovine serum albumin (BSA).

Purification of IgG Fraction of Antisera

IgG fractions of antibody prepared against the DHETs were purified from sera using protein-G affinity chromatography (Pierce Co.). The IgG bound to the protein G column was eluted with 50 mM glycine-HCl buffer, pH 2.5, and immediately neutralized with 0.5 M tris-HCl, pH 7.6. This procedure did not affect the specificity of the antibodies.

Solid Phase Competitive Enzyme-Linked Immunosorbent Assay (ELISA)

High-binding microplates were coated with protein G-purified IgG suspended in 1 M carbonate, pH 9.01 (10 μg/well; 200 μL/well final volume) and then covered with parafilm. After overnight incubation at room temperature, the welts were gently washed five times with tris buffered saline (TBS), pH 7.5, containing 0.1% tween. Non-specific sites were blocked by the addition of 0.2 mL of 5% (w/v) nonfat dry milk in TBS. After 2 hours of incubation at room temperature, they were washed three times with TBS-tween.

Standards were serially diluted with TBS (100 μL). The diluted samples (100 μL) were added to the IgG-coated plate. Approximately 50 ng of the DHET HRP conjugates were diluted with HRP-dilution buffer (100 μL) and added to the well. Following incubation for 2 hours to permit competitive binding of the molecules to the antibody, unbound material was removed by thorough washing of the wells with TBS-tween, and 150 μL of a calorimetric substrate for HRP [3,3′,5,5′ tetramethylbenzidine (TMB) and hydrogen peroxide] (Sigma Co.) was added. The plate is then incubated for 30 min, the reaction stopped by addition of 75 μL of 1 N H₂SO₄, and the absorbance at 450 nm was measured using a microtiter plate reader. Under these assay conditions, the amount of color in a well is inversely proportional to the initial concentration of the sample or the standard ligand.

Specificity of Anti-14,15- 11,12-, 8,9- and 5,6-DHETs

Anti-sera of a goat immunized with DHET-KLH conjugates showed high binding to DHET-BSA conjugates. The specificity of the 14,15-DHET ELISA was investigated using authentic DHET and a panel of eicosanoids which, based on their structure, might be anticipated to compete with 14,15-DHET for binding to antibodies against 14,15-DHET. Anti-14,15-DHET did not cross-react with 5,6-, 8,9-, 11,12- or 14,15-EET, 5,6-DHET, 15(S)HETE, 5(s)15(S)DiHETE, arachidonic acid, Thromboxane B2, PGE2, PGF₂ₐ or 6-keto-PGF₁ₐ. There was a minimal cross-reaction with 8,9- and 11,12-DHET. Specificity of 11,12-DHET was investigated using authentic 5,6-, 8,9- or 14,15-DHETs and found that there was a minimal cross-reaction with 8,9- and 14,15-DHETs. 5,6-DHET didn't cross-react with 11,12-DHET IgG.

In a typical standard graph for 14,15-DHET, the r2 value for the fit of the data to an equation describing an inverse logarithmic relationship of free 14,15-DHET to B/Bo was usually higher than 0.96. The detection limits for 14,15- and 11,12-DHET with ELISAwere ˜1 pg.

The specificity of the antibody developed against 14,15-DHET was further investigated utilizing slot blot analysis. The 14,15-DHET conjugated BSA, BSA alone and 8,9-DHET conjugated to BSA were blotted onto cellulose membrane. Slot blot analysis was carried out with anti-14,15-DHET. Though the same amount of protein is loaded to each lane (proteins were visualized by amido black staining), the antibody cross-reacted with 14,15-DHET conjugated BSA whereas the antibody failed to cross-react with 8,9-DHET which is structurally very similar to 14,15-DHET. Anti-8,9-DHET cross-reacted with both 8,9- and 14,15-DHETs. Competitive ELISA assay carried out with 5,6-DHET-HRP conjugates did not show a dose-dependent decrease of optical density at 450 nm. This result showed that 5,6-DHET IgG was not specific.

These experiments show that the unique antigenic site of a fatty acid must be located at least 10 carbons away from the carrier protein, as 8,9-DHET and 5,6-DHET IgGs were shown to be non-specific because of cross-reaction, whereas 11,12-DHET and 14,15-DHET IgGs were shown to be specific with minimal or no cross-reaction. These results allowed for the development of the fatty acid ethyl esters below.

### Example 2

### Development of the Immunoassay of Fatty Acid Ethyl Ester

Production of KLH-, BSA- and HRP-Conjugated with a Derivative of the Hydroxyl Derivative of 13-H ODE Ethyl Ester

13-HODE, a metabolite of linoleic acid, was converted to an ethyl ester with lipase (Candida antarctica) acrylic resin and ethyl alcohol in acetone (8) with modification. The ethyl ester was converted to a succinate derivative via its C13 hydroxyl group in the presence of succinic anhydride with 4-dimethylaminopyridine (4-DMAP) as a catalyst in chloroform under an argon atmosphere over 2 days (9). GC/MS analysis was performed after derivatization with MSTFA. The samples were analyzed on a HP 6890 Series GC system with a quadrupole EI/MS detector at 70 eV and a HP-5MS (capillary 30.0 m×250 μm×0.25 μm nominal 5% phenyl methyl siloxane) column. The injector temperature was 250° C. for 2 minutes, with 30° C./minute increased to 325° C. for 5 minutes. The software program was Enhanced ChemStation. The product was cross-linked to KLH, BSA, and HRP using the dicyclohexylcarbodiimide (DCC) method (10).

Antibodies Produced Against KLH-Conjugated with a Derivative of the Hydroxyl Derivative of 13-HODE Ethyl Ester.

Goats were immunized by Cocalico Biologicals, Inc. with the KLH-conjugated with a succinate derivative of the hydroxyl derivative of 13-HODE ethyl ester. The structure of the succinate derivative is shown in FIG. 2, Panel B. Antibody titers were determined by ELISA using the 13-HODE ethyl ester-conjugated BSA. The goat anti-sera recognized BSA conjugated with a derivative of the hydroxyl derivative of 13-HODE ethyl ester, whereas the sera failed to recognize BSA as evidenced by ELISA and Western blot analysis (FIG. 3).

Purification of IqG Fraction of Antisera

IgG fractions of sera were purified as described in Example 1.

Solid Phase Competitive Enzyme-Linked Immunosorbent Assay (ELISA)

ELISA was carried out with serially diluted BSA or ethyl ester of 13-HODE-conjugated BSA and ˜50 ng/100 μl of the ethyl ester of 13-HODE HRP conjugates as described in Example 1.

Western Blot Analysis

SDS-PAGE was carried out on 10% acrylamide gel. The separated proteins were electroblotted onto cellulose membrane and Western blot analyses were carried out using a HRP/ECL system.

These experiments show that antibodies successfully recognize the derivative of fatty acid ethyl esters conjugated with protein. While this example used 13-HODE ethyl ester, any fatty acid alcohol ester with a unique antigenic site located at least 10 carbons away from the carrier protein, as demonstrated by Example 1, can be successfully used to produce antibodies useful in detecting the presence of alcohol use by an individual.

Throughout this application, various publications, including Unite States patents, are referenced by author and year and patents by number. Full citations for the publications are listed below. The disclosures of these publications and patents in their entireties are hereby incorporated by reference into this application in order to more fully describe the state of the art to which this invention pertains. The invention has been used is intended to be in the nature of words of description rather than of limitation.

Obviously, many modifications and variations of the present invention are possible in the light of the above teachings. It is, therefore, to be understood that within the scope of the appended claims, the invention may be practiced otherwise than as specifically described.

TABLE 1
Correlation between amount of the succinate
product formed and ELISA results.

| | Product Peak from GC/MS* | Net Absorbance at |
|---|---|---|
| | (Integration units) | 450 nm** |
| Synthesis #1 | 0.9 × 105 | 0.51 |
| Synthesis #2 | 29 × 105 | 0.99 |

*Amount of the 13HODE ethyl ester succinate derivative produced.
**ELISA results using different BSA-HODE conjugates produced from the two separate syntheses.

### REFERENCES

1. Mattson, S. N., Schoenfeld, A. M. and Riley, E. P. (2005) Teratogenic effects of alcohol on brain and behavior. http://pubs.niaaa.nih.gov/publications/arh25-3/185-191.htm
2. Hannigan, J. H. and Armant, D. R. (2000) Alcohol in pregnancy and neonatal outcome. Semin. Neonatol. 5, 243-254.
3. Floyd, R. L., O'Connor, M. J., Sokol, R. J., Bertrand J. and Cordero, J. F. (2005) Recognition and prevention of fetal alcohol syndrome. Obstet. Gynecol. 106, 1059-1064.
4. American Academy of Pediatrics: Committee on substance abuse and Committee on children with disabilities. (1993) Fetal alcohol syndrome and fetal alcohol effects (RE9310) Pediatrics 91 (5), 1004-1006.
5. Abel, E. L. and Hannigan, J. H. (1995) Material risk factors in fetal alcohol syndrome: provocative and permissive influences. Neurotox. Teratol. 17, 445-462.
6. Bearer, C. F., Lee, S., Salvator, A. E., Minnes, S., Swick, A., Yamashita, T. and Singer, L. T. (1999) Ethyl linoleate in meconium: a biomarker for prenatal ethanol exposure. Alc. Clin. Exp. Res. 23, 487-493.
7. Bearer, C. F., Santiago, L. M., O'Riodan, M. A., Buck, K., Lee, S. and Singer, L. T. (2005) Fatty acid ethyl esters: quantitative biomarkers for maternal alcohol consumption. J. Pediatr. 146, 824-830.
8. Torres, C. F., Lessard, L. P. and Hill, C. G. (2003) Lipase-catalyzed esterification of conjugated linoleic acid with sorbitol: a kinetic study. Biotechnol. Prog. 19, 1255-1260.
9. Gu, X., Sun, M., Gugiu, G., Hazen, S., Crabb, J. W. and Salomon, R. (2003) Oxidatively truncated docosahexaenoate phospholipids: total synthesis, generation, and peptide adduction chemistry J. Org. Chem. 68, 3749-3761.
10. Tijssen, P. (1987) Conjugation of Haptens in Laboratory Techniques in Biochemistry and Molecular Biology (Eds. R. H. Burdon and P. H. van Knippenberg) Vol. 15 Practice and Theory of Enzyme Immunoassays. Pp. 279-296. Elsevier, New York, N.Y.
11. Harlow and Lane (1988) Antibodies: A laboratory Manual, Cold Spring Harbor Laboratory, Cold Spring Harbor, N.Y.
12. Borrebaeck (1992) Antibody Engineering--A practical Guide, W. H. Freeman and Co.
13. Huston et al. (1991) “Protein Engineering of Single-Chain Fv Analogs And Fusion Proteins” in Methods in Enzymology (J J Langone, ed.; Academic Press, New York, N.Y.) 203, 46-88.
14. Johnson and Bird (1991) “Construction of Single-Chain Fvb Derivatives of Monoclonal Antibodies and Their Production in Escherichia coli in Methods in Enzymology (J J Langone, ed.; Academic Press, New York, N.Y.) 203, 88-89.
15. Mernaugh and Menaugh (1995) “An Overview of Phage-Displayed Recombinant Antibodies” in Molecular Methods in Plant Pathology (R P Singh and U S Singh, eds., CRC Press Inc., Boca Raton, Fla.) pp. 359-365.
16. Johnstone & Thorpe (1982) Immunochemistry in Practice, Blackwell Scientific Publications, Oxford.

1. A method of producing an antibody recognizing a target fatty acid alcohol ester by the step of immunizing an animal with a carrier protein conjugated with a derivative of the target fatty acid alcohol ester.

2. The method as defined in claim 1, wherein the derivative of the target fatty acid alcohol ester includes a unique antigenic site at least 10 carbons away from the carrier protein.

3. The method as defined in claim 2, wherein the derivative of the target fatty acid alcohol ester is a hydroxyl derivative.

4. The method as defined in claim 3, wherein the target fatty acid alcohol ester is a fatty acid ethyl ester.

5. The method as defined in claim 4, wherein the target fatty acid ethyl ester is linoleic acid ethyl ester and the hydroxyl derivative of the target fatty acid ethyl ester is 13-hydroxyoctadeca-9,11-dienoic acid (13-HODE) ethyl ester.

6. The method as defined in claim 4, wherein the target fatty acid ethyl ester is arachidonic acid ethyl ester and the hydroxyl derivative of the target fatty acid ethyl ester is 20-hydroxy arachidonic acid (20-HETE) ethyl ester.

7. The method as defined in claim 6, wherein the target fatty acid ethyl ester is arachidonic acid ethyl ester and the hydroxyl derivative of the target fatty acid ethyl ester is 19-hydroxyeicosa-tetraenoic acid (19-HETE) ethyl ester.

8. The method as defined in claim 5, wherein the 13-HODE ethyl ester is synthesized with 13-HODE and ethanol using lipase.

9. The method as defined in claim 6, wherein the 20-HETE ethyl ester is synthesized with 20-HETE and ethanol using lipase.

10. The method as defined in claim 8, wherein the 13-HODE is enzymatically synthesized using linoleic acid as a substrate of the enzyme.

11. The method as defined in claim 9, wherein the 20-HETE is enzymatically synthesized using arachidonic acid as a substrate of the enzyme.

12. The method as defined in claim 10, wherein the enzyme is lipoxygenase.

13. The method as defined in claim 11, wherein the enzyme is cytochrome P450 4A.

14. A method of estimating alcohol consumption of an individual by the step of quantitating levels of molecules which bind to antibodies produced with a derivative of a target fatty acid alcohol ester conjugated to a carrier protein in a biological sample obtained from the individual.

15. The method as defined in claim 14, wherein the derivative of the target fatty acid alcohol ester includes a unique antigenic site at least 10 carbons away from the carrier protein.

16. The method of claim 15, wherein the derivative of the target fatty acid alcohol ester is 13-HODE ethyl ester.

17. The method as defined in claim 15, wherein the derivative of the target fatty acid alcohol ester is 20-HETE ethyl ester.

18. The method as defined in claim 15, wherein said quantitating step is further defined as quantitating levels of molecules to determine prenatal exposure of an infant to alcohol.

19. The method as defined in claim 18, wherein the biological sample is mecodium from the infant.

20. The method as defined in claim 15, wherein said quantitating step is further defined as quantitating levels of molecules to determine recent alcohol use by the individual.

21. The method as defined in claim 15, wherein said quantitating step is performed before and after treatment of a mammal with an alcohol-dependency lowering drug to estimate the effect of the drug.

22. A kit for estimating alcohol consumption of an individual comprising means for quantitating levels of molecules which bind to antibodies produced with a derivative of a target fatty acid alcohol ester conjugated to a carrier protein in a biological sample obtained from the individual.

23. The kit as defined in claim 22, wherein the derivative of the target fatty acid alcohol ester includes a unique antigenic site at least 10 carbons away from the carrier protein.

24. An antibody recognizing a target fatty alcohol ester produced by the method of claim 1.

25. The antibody as defined in claim 24, wherein the derivative of the target fatty acid alcohol ester includes a unique antigenic site at least 10 carbons away from the carrier protein.
